(19) United States
(12) Patent Application Publication
(10) Pub. No.: US 2023/0170057 A1
(43) Pub. Date: Jun. 1, 2023

(54) ASSOCIATING COMPLEX DISEASE SCORES AND BIOMARKERS WITH MODEL PARAMETERS USING MODEL INVERSE SURROGATES

(71) Applicant: INTERNATIONAL BUSINESS MACHINES CORPORATION, ARMONK, NY (US)

(72) Inventors: James R. Kozloski, New Fairfield, CT (US); Viatcheslav Gurev, Bedford Hills, NY (US); Tim Rumbell, Raleigh, NC (US)

(21) Appl. No.: 17/456,966

(22) Filed: Nov. 30, 2021

(51) Int. Cl. G16C 20/30; G16H 50/20; G06N 3/08; G16C 20/50; G16C 20/70

(52) U.S. Cl. CPC G16C 20/30; G16H 50/20; G06N 3/08; G16C 20/50; G16C 20/70

(57) ABSTRACT

A method, computer system, and a computer program product for model inversion is provided. The present invention may include training a generator of a generative adversarial network to sample a distribution of input parameters of a mechanistic model. The present invention may include generating a distribution of parameters for the mechanistic model. The present invention may include simulating the mechanistic model with the distribution of parameters.

### BACKGROUND

The present invention relates generally to the field of computing, and more particularly to inverting mechanistic models.

Mechanistic modeling has been increasingly applied within at least biophysics, medicine, and/or pharmacology, and across various stages of drug development and/or therapeutic areas. Mechanistic models that may be utilized, may include, but are not limited to including, models which may be utilized in associating a drug's mechanisms of action with systems level responses in at least cells, tissues, organ systems, and/or patient populations as a whole and/or models which may be utilized in at least associating mechanisms of drug absorption, distribution, metabolism, and/or excretion in the body and/or body tissue with the appropriate dosing and/or expected concentrations of a drug at a target in a patient's body.

Accordingly, mechanistic models may be instrumental in at least determining drug dosages and/or which drug may be most beneficial for a patient population and/or individual patient.

### SUMMARY

Embodiments of the present invention disclose a method, computer system, and a computer program product for inverting mechanistic models. The present invention may include training a generator of a generative adversarial network to sample a distribution of input parameters of a mechanistic model. The present invention may include generating a distribution of parameters for the mechanistic model. The present invention may include simulating the mechanistic model with parameters sampled from the distribution of parameters.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flowchart illustrating a process for model inversion according to at least one embodiment;

FIG. 3 is a training process utilized by the model inversion program in generating a distribution of parameters according to at least one embodiment;

FIG. 4 is a block diagram of internal and external components of computers and servers depicted in FIG. 1 according to at least one embodiment;

FIG. 5 is a block diagram of an illustrative cloud computing environment including the computer system depicted in FIG. 1, in accordance with an embodiment of the present disclosure; and

FIG. 6 is a block diagram of functional layers of the illustrative cloud computing environment of FIG. 5, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for model inversion. As such, the present embodiment has the capacity to improve the technical field of mechanistic models by incorporating conditioning auxiliary variables into a regularized generative adversarial network structure. More specifically, the present invention may include training a generator of a generative adversarial network to sample a distribution of input parameters of a mechanistic model. The present invention may include generating a distribution of parameters for the mechanistic model. The present invention may include simulating the mechanistic model with parameters sampled from the distribution of parameters.

As described previously, mechanistic modeling has been increasingly applied within at least biophysics, medicine, and/or pharmacology, and across various stages of drug development and/or therapeutic areas. Mechanistic models that may be utilized, may include, but are not limited to including, models which may be utilized in associating a drug's mechanisms of action with systems level responses in at least cells, tissues, organ systems, and/or patient populations as a whole and/or models which may be utilized in at least associating mechanisms of drug absorption, distribution, metabolism, and/or excretion in the body and/or body tissue with the appropriate dosing and/or expected concentrations of a drug at a target in a patient's body.

Accordingly, mechanistic models may be instrumental in at least determining drug dosages and/or which drug may be most beneficial for a patient population and/or individual patient.

Therefore, it may be advantageous to, among other things, train a generator of a generative adversarial network to sample a distribution of input parameters of a mechanistic model, generate a distribution of parameters for the mechanistic model, and simulate the mechanistic model with parameters sampled from the distribution of parameters.

According to at least one embodiment, the present invention may improve solutions to stochastic inverse problems (e.g., populations of model's problem) with mechanistic models using informative auxiliary variables in conditioning a target distribution of outputs. The informative auxiliary variables may not be derived from model input parameters nor model output domains.

According to at least one embodiment, the present invention may improve the incorporation of measures present in experimental data sets into mechanistic model-based analyses by incorporating conditioning auxiliary variables into a regularized generative adversarial network (GAN) structure.

According to at least one embodiment, the present invention may improve solutions to stochastic inverse problems (e.g., populations of model's problems) with mechanistic models using an adversarial approach and incorporating multiple discriminators as optimization constraints.

According to at least one embodiment, the present invention may improve mechanistic modeling by using a conditional regularized generative adversarial network (cr-GAN) trained on a standard pharmacokinetic (PK) model with conditional variables associated with other measurements utilized in parametrization of a physiology-based component in a physiology-based pharmacokinetic (PBPK) model, such as, but not limited to, dosing and concentrations of secondary drugs in drug-drug interactions (DDI), concentrations of bodily proteins, patient conditions, amongst other physiology-based conditional variables. This may improve, at least, training, transfer learning, and/or inference over the cr-GAN for drug PK, which may reduce cost and/or context-specific construction of the physiology-based component in a physiology-based pharmacokinetic (PBPK) model.

According to at least one embodiment, the present invention may improve assessing bioequivalence of compounds using the cr-GAN architecture to infer PK model parameters in virtual bioequivalence (VBE) assessments based on training the cr-GAN with conditioning variable from drug formulation embeddings.

According to at least one embodiment, the present invention may improve the ability to model different types of objects as inputs and outputs of mechanistic models using convolutional neural networks for signal and/or image processing, graph convolutional networks, and/or entity embeddings of categorial variables which may be incorporated into the GAN.

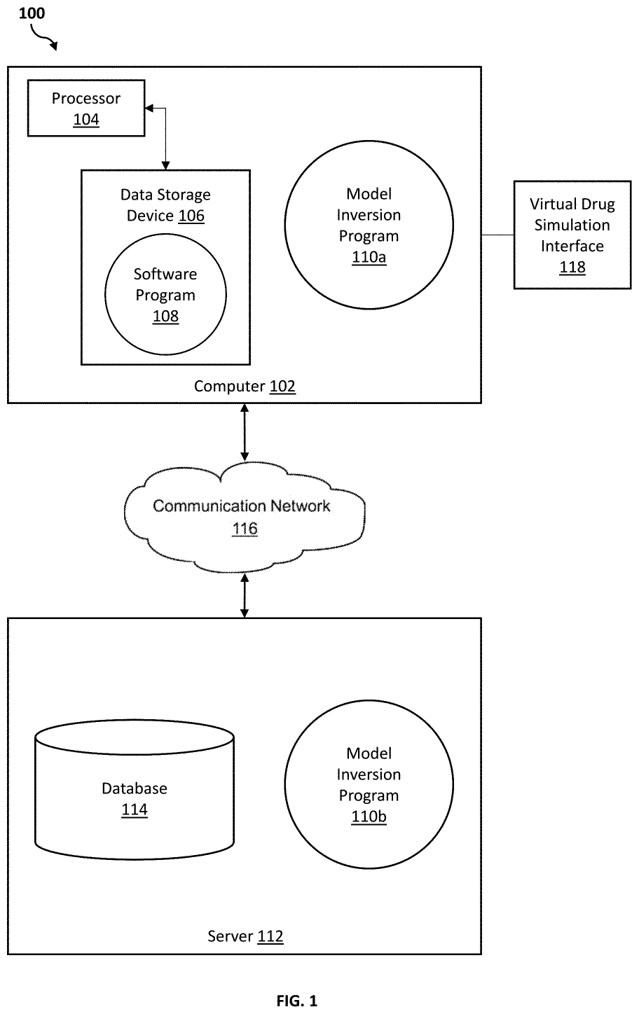

Referring to FIG. 1, an exemplary networked computer environment 100 in accordance with one embodiment is depicted. The networked computer environment 100 may include a computer 102 with a processor 104 and a data storage device 106 that is enabled to run a software program 108 and a model inversion program 110a. The networked computer environment 100 may also include a server 112 that is enabled to run a model inversion program 110b that may interact with a database 114 and a communication network 116. The networked computer environment 100 may include a plurality of computers 102 and servers 112, only one of which is shown. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

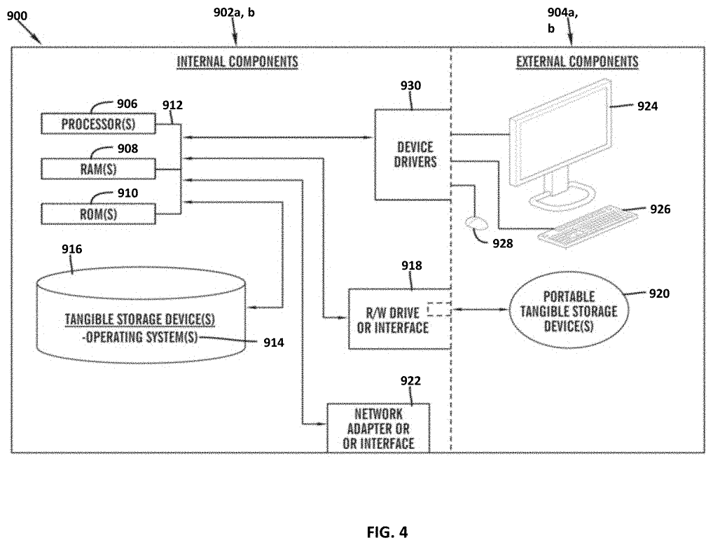

The client computer 102 may communicate with the server computer 112 via the communications network 116. The communications network 116 may include connections, such as wire, wireless communication links, or fiber optic cables. As will be discussed with reference to FIG. 4, server computer 112 may include internal components 902a and external components 904a, respectively, and client computer 102 may include internal components 902b and external components 904b, respectively. Server computer 112 may also operate in a cloud computing service model, such as Software as a Service (SaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the model inversion program 110a, 110b may interact with a database 114 that may be embedded in various storage devices, such as, but not limited to a computer/mobile device 102, a networked server 112, or a cloud storage service.

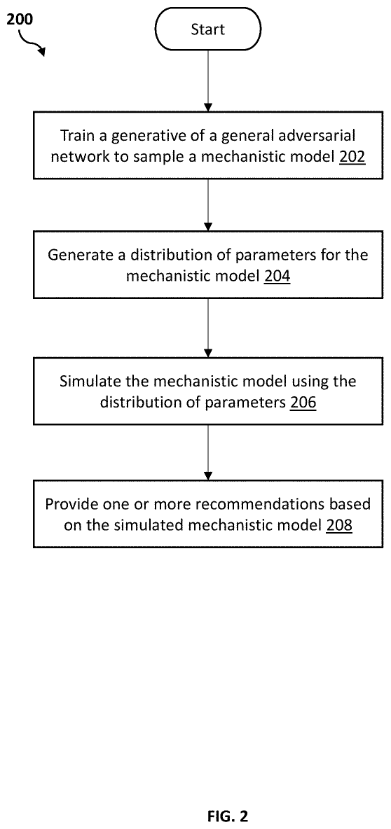

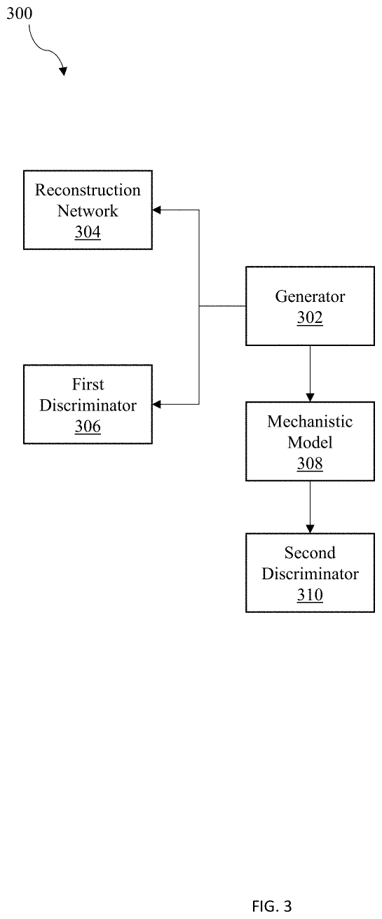

According to the present embodiment, a user using a client computer 102 or a server computer 112 may use the model inversion program 110a, 110b (respectively) to generate samples from a distribution of parameters for a mechanistic model associated with an auxiliary variable. The model inversion method is explained in more detail below with respect to FIGS. 2 and 3.

Referring now to FIG. 2, an operational flowchart illustrating the exemplary model inversion process 200 used by the model inversion program 110a and 110b (hereinafter model inversion program 110) according to at least one embodiment is depicted.

At 202, the model inversion program 110 trains a generator of a generative adversarial network. The model inversion program 110 may train the generator of the generative adversarial network (GAN) to sample a distribution of input parameters of a mechanistic model. The GAN may be a conditional regularized Generative Adversarial Network (cr-GAN) for constrained optimization, in which the generator is trained to sample the distribution of input parameters of the mechanistic model.

The mechanistic model may be a non-invertible mathematical model utilized in drug development, such as, but not limited to, Quantitative Systems Pharmacology (QSP) models and/or Pharmacokinetic (PK) models. The mechanistic models may be non-invertible models, wherein more than one parameterization of the model (i.e., different parameter sets) may result in model outputs which may be equal. Accordingly, a specific parameter set which may be utilized in generating an output of the non-invertible model may not be explicitly computed, using standard mathematical inversion methods, from the output of the model.

QSP models may be utilized in at least target and/or disease-related biomarker identification, hypothesis testing, prediction of efficacy and/or toxicity, and determining a drug based on complications of biological processes. The biological processes may include physiological consequences of a disease, a specific disease pathway (e.g., signal transduction and/or up/down regulation of a pathway), and/or biological processes related to omics (i.e., genomics, proteomics, metabolomics), among other things. QSP models may be utilized in describing mechanistic connections between biological pathways and objectively measurable outcomes and/or biomarkers.

PK models may be mathematical tools that enable simulation of drug concentration levels in blood prior to administration. PK models may model a time course of a given drug concentration in the body, in which the model parameters may be rates of drug distribution and elimination. The PK model may be a two-compartment PK model such that the time course of the given drug concentration in the body may be modeled in both a central compartment, such as blood plasma, and a peripheral compartment, such as body tissue. A two-compartment PK model may enable monitoring of biphasic decay over time.

Mechanistic models may utilize data from clinical datasets and/or pre-clinical datasets which may include observations that map to either parameters and/or outputs of the mechanistic model. The observations may be measurements from a patient population and/or individual patient, as well as auxiliary variables (e.g., conditioning variables, auxiliary random variables) which do not map to either parameters and/or outputs of the mechanistic model. The clinical datasets and/or pre-clinical datasets may be utilized as input parameters for the mechanistic model.

For example, input parameters that may be utilized in the QSP models for drug development may include, but are not limited to including, bioavailability, volume of distribution and distribution phase, clearance, half-life and/or protein binding of drugs, ion channel model parameters, receptor binding affinities, time constants of biological processes such as membrane kinetics and signal propagation, structural parameters such as brain structural volumes, thickness, connectivity and/or heart chamber morphology and volume, and metabolite concentrations, amongst other input parameters. Bioavailability may depend, at least in part, on a drug's formulation. A drug that may be significantly metabolized as it first passes through the liver may exhibit a marked first-pass effect, which may reduce the effective oral absorption of the drug. Accordingly, a reduction in first-pass effect may cause an increase in oral drug absorption. The volume of distribution of a drug may determine a plasma concentration after a loading dose. The distribution phase may be the time taken for a drug to distribute from plasma to periphery. Drug levels drawn before completions of a long distribution phase may not reflect levels of pharmacologically active drugs at a site of action. Clearance may be either renal or nonrenal, for most drugs measured, and may be independent of plasma drug concentration, such that a change in dose may be reflected by a similar change in plasma level. The half-life of a drug may depend on at least its volume of distributions and clearance which may determine the time taken to reach a steady state level. The protein binding of drugs may refer to the degree in which medications attach to proteins in the blood for a given drug/medication as well as the free drug level. PK models may utilize fewer auxiliary variables (e.g., conditioning variables, auxiliary random variables) than QSP models. For example, PK models may not utilize disease scores, as will be explained in more detail below, but may utilize biomarkers such as concentration of a drug in a body tissue.

The auxiliary variables (e.g., conditioning variables, auxiliary random variables) may be associated with the mechanistic model's parameter space through co-occurrence with data that maps to model outputs. The auxiliary variables (e.g., conditioning variables, auxiliary random variables) may influence the observations of the clinical datasets and/or pre-clinical datasets which may be specific to a patient population and/or individual patient but the mechanistic model may not capture the auxiliary variables (e.g., conditioning variables, auxiliary random variables as outputs. Auxiliary variables may include, but are not limited to including, disease metrics (e.g., complex disease metrics, disease scores) and/or biomarkers (e.g., biomarker scores). The model inversion program 110 may utilize the auxiliary variables in conditioning the cr-GAN, such that the cr-GAN, when presented with a set of biological phenotypes and a desired disease metric, may sample from a distribution of parameters for the mechanistic model that map to biological phenotype data subject to the disease metric.

Disease metrics (e.g., complex disease metric, disease scores) may include disease metrics such as, but not limited to, Unified Parkinson Disease Rating Scale (UPRDS) scores, Unified Huntington's Disease Rating Scale (UHDRS) scores, Eczema Area and Severity Index (EAST) scores amongst other disease scores. Disease metrics (e.g., complex disease metric, disease scores) may be utilized in assessing patient responses. Disease metrics (e.g., complex disease metrics, disease scores) may be measures for an individual patient, while a distribution of disease metrics may be utilized for a patient population. For example, to model two patient populations, the model inversion program 110 may sample from each distribution of complex disease metrics and may apply each sample as an auxiliary variable (e.g., conditioning variable, auxiliary random variable) to the cr-GAN. In this example, the model inversion program 110 may generate two distributions of model parameters, wherein each of the two distributions of model parameters corresponds to one of the two patient populations.

Biomarkers (e.g., biomarker scores) may be any measurable substance in an organism whose presence may be indicative of some phenomenon such as disease, infection, and/or environmental exposure. With respect to mechanistic models, a drug may affect the measurable substance and biomarkers which may alter measurements of at least the drug's effect on the bodily systems such as disease phenotypes, absorption, distribution, metabolism, and/or excretion from a patient's body. For example, biomarkers (e.g., biomarker scores) may be a signature in an Electroencephalography (EEG), such as, but not limited to a spectral power in a beta band and/or blood concentration of a biomarker, such as, but not limited to, a neuro filament-light chain.

As will be explained in more detail below, the model inversion program 110 may pass the auxiliary variables (e.g., conditioning variables, auxiliary random variables) to both the generator and at least one of the two or more discriminators, such that the generator and the at least one discriminator may each access the auxiliary variables while undergoing training. The at least one discriminator may attempt to distinguish actual auxiliary variables from imitation auxiliary variables generated by running generated parameters through the mechanistic model, given the auxiliary variables (e.g., conditioning variables, auxiliary random variables).

In an embodiment, the model inversion program 110 may utilize a GAN architecture which may be comprised of at least one generator, at least two discriminators, along with a reconstruction network which may reproduce base variables (e.g., base distribution) from the output the at least one generator and/or the mechanistic model, as will be explained in more detail below. A first discriminator may distinguish between samples from a joint distribution and samples which may be generated by the at least one generator. The samples generated by the at least one generator may be forwarded through the mechanistic model and/or augmented with a conditioning variable (e.g., auxiliary variables, auxiliary random variables), for which a standard conditional loss may be maximized. A second discriminator may distinguish between samples from prior model parameters and samples which may be generated by the at least one generator, for which a standard loss may be maximized. The reconstruction network may reproduce the base variables (e.g., base distribution) from samples which may be generated by the at least one generator, for which a squared loss may be minimized. As will be explained in more detail below with respect to step 204, the generator may generate a distribution of parameters from at least, the base variables (e.g., base distribution), augmented with the conditioning variable (e.g., auxiliary variables, auxiliary random variables), for which a weighted sum loss may be minimized.

In an embodiment, the minimization of divergences between the generated distributions of model parameters and the prior distribution of parameters for the mechanistic model, subject to equality of distribution of mechanistic model output to the target, may be solved within a parametric model of parameter density. The model inversion program 110 may utilize at least the method of Lagrangian multipliers, mix of parametric models with deep learning networks, amongst other methods in this embodiment.

For example, an individual's smoking status may affect the way in which a drug is metabolized and/or taken up by a target tissue. This may be reflected in different distributions of target tissue concentrations measured from patients with different smoking statuses. However, the transformation of one distribution to another distribution may be nonlinear. In this example, the parameters of the PK model capable of generating these two distributions of parameters may be represented by nontrivial inversions of the data distributions to those parameters. Accordingly, the cr-GAN in this example may be utilized to learn simultaneously the inversion from the data of the two populations to the different distributions of model parameters given the smoking status of the patients represented by each distribution.

At 204, the model inversion program 110 generates a distribution of parameters. The model inversion program 110 may generate the distribution of parameters for the mechanistic model using the trained cr-GAN. The distribution of parameters generated by the model inversion program 110 may depend on the mechanistic model and/or auxiliary variables. As will be explained in more detail below, the distribution of parameters generated for a QSP model may be utilized in determining a drug, while the distribution of parameters generated for a PK model may be utilized in determining a drug dose. The model inversion program 110 may utilize the auxiliary variables (e.g., conditioning variables, auxiliary random variables) of each patient in a patient population, such that the model inversion program 110 may sample from a distribution of auxiliary variables (e.g., conditioning variables, auxiliary random variables) for the patient population. The model inversion program 110 may sample from one or more patient populations and apply a sample of each the distribution of auxiliary variables from the one or more patient populations as the auxiliary variables (e.g., conditioning variables, auxiliary random variables) to the cr-GAN.

In the QSP model, the model inversion program 110 may utilize the trained cr-GAN to generate the distribution of parameters for the QSP model associated with the auxiliary variables (e.g., conditioning variables, auxiliary random variables) for a patient population and/or individual patient. In the QSP model, the auxiliary variables (e.g., conditioning variables, auxiliary random variables) may be the disease metric (e.g., complex disease metric, disease scores) and/or biomarkers (e.g., biomarker scores) of the dataset for the patient population and/or the individual patient. The distribution of parameters generated by the model inversion program 110 for the QSP model may include at least a range of mechanistic causes for a disease state and/or patient measure which may have not been captured in the QSP model input parameters and/or the QSP output domains.

In the PK model, the model inversion program 110 may utilize the trained cr-GAN to generate the distribution of parameters for the PK model associated with the auxiliary variables (e.g., conditioning variables, auxiliary random variables) for a patient population and/or individual patient. In the PK model the auxiliary variables (e.g., conditioning variables, auxiliary random variables) may be the biomarkers (e.g., biomarker scores) of the dataset for the patient population and/or the individual patient. The distribution of parameters generated by the model inversion program 110 for the PK model may include at least one or more ranges associated with the biomarker (e.g., biomarker scores) of the dataset. The one or more ranges associated with the biomarker may include, but are not limited to including, one or more of, a range of drug absorption levels, a range of drug distribution levels, a range of drug metabolism levels, and/or a range of drug excretion levels of the patient population and/or the individual patient. The distribution of parameters may also include an expected drug concentration at a target body tissue. The expected drug concentration at the target body tissue may be based on at least drug concentrations at the target body tissue for patient populations from the dataset with similar biomarkers.

For example, the model inversion program 110 may utilize a two-compartment PK model. The structure of the two-compartment PK model may include amount of a drug in a central compartment (i.e., blood plasma) and amount of the drug in a peripheral compartment (i.e., body tissue). In this example, the model inversion program 110 may model an intravenous administration of a drug dose directly into the central compartment (i.e., blood plasma), which may exhibit a biphasic decay over time which may be fit with a two-exponential decay curve.

At 206, the model inversion program 110 simulates the mechanistic model with the distribution of parameters generated using the trained cr-GAN. The model inversion program 110 may utilize one or more virtual drugs and/or one or more virtual drug doses in simulating the mechanistic model with the distribution of parameters generated.

The model inversion program 110 may simulate the QSP model with the distribution of parameters using one or more virtual drugs. The model inversion program 110 may simulate the one or more virtual drugs on the parameterized QSP model population. The model inversion program 110 may quantify one or more expected outcomes of the simulation. The one or more expected outcomes may be dependent on at least the one or more virtual drugs applied and/or the distribution of parameters generated by cr-GAN. As will be explained in more detail below, the one or more outcomes of the simulation may be utilized by the model inversion program 110 in determining which drug to administer to a patient population and/or individual patient.

For example, QSP models may generate features that are sampled from a distribution of biomarkers and/or disease/normal phenotypes of a patient population, such as, electrophysiological biomarkers, medical imaging biomarkers, and/or blood biomarkers. In this example, model inversion program 110 may generate a distribution of parameters for the QSP model for a disease population. The distribution of parameters may be modified in the simulation of the QSP model by the model inversion program 110 to represent treatments of one or more virtual drugs. The model inversion program 110 may simulate these distributions of parameters for the one or more virtual drugs through the QSP model to ascertain whether or not the produced, new biomarker features, may be closer to the normal (e.g., healthy) distribution of parameters according to one or more distance metrics utilized by the model inversion program 110, such as, but not limited to a Jensen-Shannon distance (e.g., JS distance). The model inversion program 110 may simulate the QSP model for each of the one or more virtual drugs which may enable the model inversion program 110 to determine which of the one or more virtual drugs minimized the distance between the distribution of parameters for the disease population and the normal (e.g., healthy) distribution to the greatest degree.

The model inversion program 110 may simulate the PK model with the distribution of parameters using one or more virtual drug doses. The model inversion program 110 may utilize the one or more virtual drug doses in determining at least a targeted tissue concentration and/or blood concentration of the virtual drug. As will be explained in more detail below, the targeted tissue concentration and/or the blood concentration of the virtual drug may be utilized by the model inversion program 110 in determining whether the target tissue concentration and/or the blood concentration of the virtual drug may be within a well-tolerated range of the patient population and/or individual patient associated with the biomarker.

For example, the model inversion program 110 may simulate the PK model with the distribution of parameters using the one or more virtual drug doses. In this example, the model inversion program 110 may vary the drug dose and simulate the different target tissue concentration distributions for each of the one or more virtual drug doses. The model inversion program 110 may utilize a distance metric in determining which of the one or more virtual drug doses is closest to the target tissue concentration.

The model inversion program 110 may display simulations of the mechanistic model in a virtual drug simulation interface 118. The virtual drug simulation interface 118 may be displayed by the model inversion program 110 in at least an internet browser, dedicated software application, and/or as an integration with a third party software application. The model inversion program 110 may display simulation of the mechanistic model in the virtual drug simulation interface using one or more graphs. The one or more graphs may represent multi-variable distributions of measurement features of at least, disease patient populations, normal/healthy patient populations, and/or simulated mechanistic models of the one or more virtual drugs and/or one or more virtual drug dosages. For example, the model inversion program 110 may display an overlap of the simulated mechanistic model and the normal/healthy patient population using one or more distance metrics. The display may enable the model inversion program 110 to rank the one or more virtual drugs and/or drug doses simulated for the mechanistic models.

At 208, the model inversion program 110 provides one or more recommendations for administration based on the simulation of the mechanistic models. The one or more recommendations for administration may include recommending a drug and/or recommending a drug dose for the patient population and/or the individual patient.

The model inversion program 110 may recommend a drug based on at least a mapping of associated QSP model outcomes with the disease metric (e.g., complex disease metric, disease scores) or biomarkers of disease that the QSP model can replicate in the patient population and/or the individual patient for each of the one or more virtual drugs. The mapping of the associated QSP model may be utilized by the model inversion program 110 in determining whether the endpoint can be achieved by each of the one or more virtual drugs utilized in the simulation. The model inversion program 110 may recommend a drug based on the mapping of the corresponding virtual drug which maximizes the probability of the patient population and/or individual patient being shifted to a healthy state.

The model inversion program 110 may recommend the drug dose based on one or more simulations of the PK model which result in the target tissue and/or blood concentrations that may be expected to be well-tolerated by the patient population and/or individual patient. The model inversion program 110 may adjust the dose of the drug until the expected drug concentration may be well tolerated by the patient population and/or individual patient as indicated by the population of PK models associated with the resulting drug concentrations and biomarkers. The model inversion program 110 may adjust the dose of the drug using one or more different dosing regimens, wherein the one or more dosing regimens may adjust for at least drug decay over time. The recommended drug dose may have a desired concentration over an extended period of time. The model inversion program 110 may simulate the one or more dosing regimens to identify a minimum risk dosing regimen for the patient population and/or individual patient.

For example, a user of the model inversion program 110 may utilize a population of models in which the user can simulate through the model inversion program 110 one or more dosing regimens and identify a full spectrum of probabilities associated with those dosing regimens. This may allow the user to make a more informed decision with respect to at least dosing concentrations and/or frequencies based on the assessment of probabilities presented by the model inversion program 110.

Referring now to FIG. 3, a training process 300 which may be utilized by the model inversion program 110 in generating a distribution of parameters according to one embodiment is depicted.

The training process 300 depicted may be utilized in training the generator 302 of the generative adversarial network (GAN) which may in turn be utilized in generating the distribution of parameters, as detailed in step 204 above. A first stage of the training process may utilize at least a generator 302, a reconstruction network 304, and a first discriminator 306. The generator 302, the reconstruction network 304, the first discriminator 306, and a second discriminator 310, may all be feedforward neural networks, such that the connections between nodes may not form a cycle. Although the first discriminator 306 and the second discriminator 310 of FIG. 3 are depicted separately they may be the same discriminator which may merely function differently based on input and/or the first discriminator 306 and the second discriminator 310 may be two separate discriminators as depicted.

In the first stage of the training process, the generator 302 may sample from a distribution of model parameters from the base variables (e.g., base distribution) of the mechanistic model 308. The mechanistic model may be a QSP model and/or a PK model. The distribution of model parameters may be a Gaussian base distribution which may include an equal number of measurements above and below a mean value for a patient population. The generator 302 may also receive as input one or more auxiliary variables (e.g., conditioning variables, auxiliary random variables), such as, but not limited to, biomarkers (e.g., biomarker scores) and/or disease metrics (e.g., complex disease metrics, disease scores) associated with the patient population from with the distribution of model parameters may be sampled. The generator 302 may augment the distribution of parameters using the one or more auxiliary variables (e.g., conditioning variables, auxiliary random variables) to generate model parameters. The model parameters generated by the generator 302 may be utilized as input for the reconstruction network 304 and/or the first discriminator 306.

The reconstruction network 304 may utilize the model parameters generated by the generator in reproducing the base variables (e.g., base distribution) of the mechanistic model 308 in which the distribution of model parameters may have been sampled from, for which a squared loss may be minimized. Reconstruction loss may be based on a measure of the similarity between the base variables (e.g., base distribution) and the model parameters generated by the generator 302. The model inversion program 110 may utilize the reconstruction loss to force the generator 302 towards configurations of indistinguishable data which may be utilized by the model inversion program 110 in generating the model parameters for the mechanistic model 308 as will be explained in more detail below with respect to the second stage of the training process. The model inversion program 110 may utilize one or more adaptive learning rate optimization algorithms which may be utilized in at least adjusting attributes of the GAN such as weighting and/or learning rate which may reduce the reconstruction loss.

The first discriminator 306 may utilize the model parameters generated by the generator 302 and the prior distribution of parameters for the mechanistic model 308 as input. The first discriminator 306 may distinguish between the model parameters generated by the generator 302 and the prior distribution of parameters for the mechanistic model 308, for which a standard loss (e.g., adversarial loss) may be maximized. Adversarial loss from the first discriminator 306 may be utilized by the model inversion program 110 in minimizing a divergence between the model parameters generated by the generator 302 and the prior distribution of parameters for the mechanistic model 308. The model inversion program 110 may utilize the adversarial loss to force the generator 302 towards configurations of indistinguishable data which may be utilized by the model inversion program 110 in generating the model parameters for the mechanistic model 308 as will be explained in more detail below with respect to the second stage of the training process. The model inversion program 110 may utilize one or more adaptive learning rate optimization algorithms in at least adjusting attributes of the GAN such as weighting and/or learning rate which may reduce the adversarial loss.

The second stage of the training process may utilize at least a generator 302, a mechanistic model 308, and a second discriminator 310. The model inversion program 110 may forward the model parameters generated by the generator 102 through the mechanistic model 308, which may be a QSP model and/or PK model. The model inversion program 110 may forward the model parameters generated by the generator 102 through the mechanistic model based on at least the adversarial loss and/or the reconstruction loss detailed above with respect to the first stage of the training process being within an acceptable divergence between the model parameters generated by the generator 302 and the prior distribution of parameters for the mechanistic model 308. The mechanistic model 308 may utilize the model parameters generated by the generator 302 as input and generate a model output. The model inversion program 110 may augment the model output with the one or more auxiliary variables (e.g., conditioning variables, auxiliary random variables) and may utilize the augmented model output as input for the second discriminator 310.

The second discriminator 310 may utilize the augmented model output and sample data as input. The sample data may be a distribution of patient measures derived from target distribution of model outputs conditioned on the one or more auxiliary variables (e.g., conditioning variables, auxiliary random variables), the target distribution of model outputs may be at least a targeted tissue concentration and/or blood concentrations of a virtual drug in a PK model and/or a targeted outcome of a virtual drug in a QSP model for the patient population. The second discriminator 310 may distinguish between the augmented model output from the mechanistic model 308 and the sample data, for which a standard conditional loss is maximized. The standard conditional loss may be utilized by the model inversion program 110 in minimizing a divergence between the model output of the mechanistic model augmented with the one or more auxiliary variables (e.g., conditioning variables, auxiliary random variables) and the sample data.

As explained in more detail above with respect to step 206, the model inversion program 110 may utilize the distribution of parameters in simulating the mechanistic model 308. The model inversion program 110 may utilize the distribution of parameters in simulating the mechanistic model based on the standard conditional loss being within an acceptable divergence between the augmented model output and the sample data as determined by the second discriminator 310.

It may be appreciated that FIGS. 2 and 3 provide only an illustration of one embodiment and do not imply any limitations with regard to how different embodiments may be implemented. Many modifications to the depicted embodiment(s) may be made based on design and implementation requirements.

FIG. 4 is a block diagram 900 of internal and external components of computers depicted in FIG. 1 in accordance with an illustrative embodiment of the present invention. It should be appreciated that FIG. 4 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902 a, b and external components 904 a, b illustrated in FIG. 4. Each of the sets of internal components 902 a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the model inversion program 110a in client computer 102, and the model inversion program 110b in network server 112, may be stored on one or more computer-readable tangible storage devices 916 for execution by one or more processors 906 via one or more RAMs 908 (which typically include cache memory). In the embodiment illustrated in FIG. 4, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902 a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the model inversion program 110a and 110b can be stored on one or more of the respective portable computer-readable tangible storage devices 920, read via the respective R/W drive or interface 918 and loaded into the respective hard drive 916.

Each set of internal components 902 a, b may also include network adapters (or switch port cards) or interfaces 922 such as a TCP/IP adapter cards, wireless wi-fi interface cards, or 3G or 4G wireless interface cards or other wired or wireless communication links. The software program 108 and the model inversion program 110a in client computer 102 and the model inversion program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the model inversion program 110a in client computer 102 and the model inversion program 110b in network server computer 112 are loaded into the respective hard drive 916. The network may comprise copper wires, optical fibers, wireless transmission, routers, firewalls, switches, gateway computers and/or edge servers.

Each of the sets of external components 904 a, b can include a computer display monitor 924, a keyboard 926, and a computer mouse 928. External components 904 a, b can also include touch screens, virtual keyboards, touch pads, pointing devices, and other human interface devices. Each of the sets of internal components 902 a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

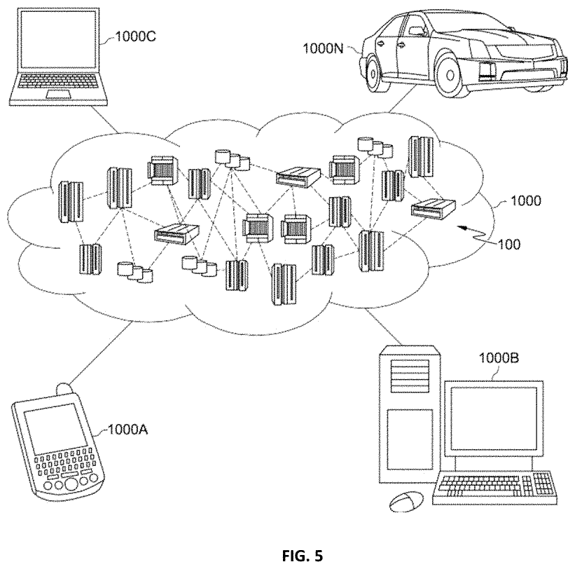

Referring now to FIG. 5, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 5 are intended to be illustrative only and that computing nodes 100 and cloud computing environment 1000 can communicate with any type of computerized device over any type of network and/or network addressable connection (e.g., using a web browser).

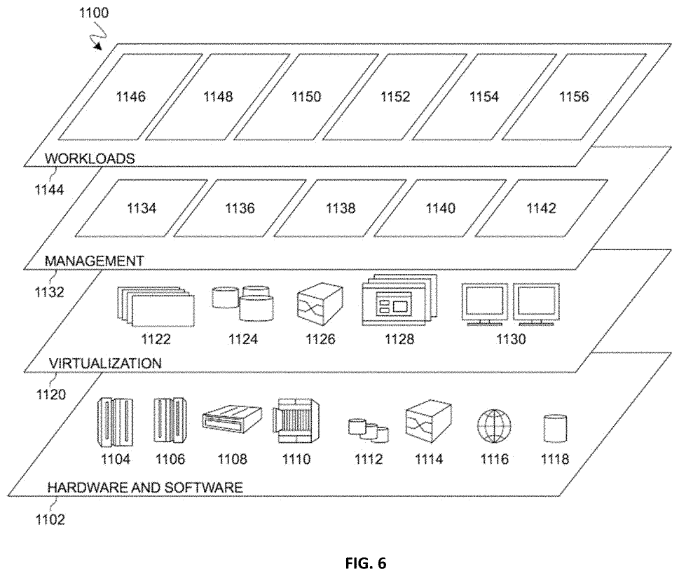

Referring now to FIG. 6, a set of functional abstraction layers 1100 provided by cloud computing environment 1000 is shown. It should be understood in advance that the components, layers, and functions shown in FIG. 6 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and model inversion 1156. A model inversion program 110a, 110b provides a way to generate a distribution of parameters with an associated auxiliary variable.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

The present disclosure shall not be construed as to violate or encourage the violation of any local, state, federal, or international law with respect to privacy protection.

What is claimed is:

1. A method for model inversion, the method comprising:
training a generator of a generative adversarial network to sample a distribution of input parameters of a mechanistic model, wherein the mechanistic model is a QSP model;
generating a distribution of parameters for the QSP model; and
simulating the QSP model with the distribution of parameters.

2. The method of claim 1, wherein the distribution of parameters for the QSP model are associated with an auxiliary variable.

3. The method of claim 2, wherein the auxiliary variable is a disease metric for a patient population.

4. The method of claim 1, wherein simulating the QSP model with the distribution of parameters further comprises:
using one or more virtual drugs.

5. The method of claim 4, further comprising:
displaying one or more expected outcomes of the QSP model for each of the one or more virtual drugs in a virtual drug simulation interface.

6. The method of claim 1, further comprising:
providing a drug recommendation for a patient population based on the QSP model simulation.

7. The method of claim 1, wherein training the generator of the generative adversarial network further comprises:
utilizing at least two discriminators and a reconstruction network in training the generator.

8. The method of claim 7, wherein one of the at least two discriminators distinguishes from samples from a joint distribution and samples generated by the generator.

9. The method of claim 7, wherein one of the at least two discriminators distinguishes from samples from prior model parameters and samples generated by the generator.

10. A method for model inversion, the method comprising:
training a generator of a generative adversarial network to sample a distribution of input parameters of a mechanistic model, wherein the mechanistic model is a PK model;
generating a distribution of parameters for the PK model; and
simulating the PK model with the distribution of parameters.

11. The method of claim 10, wherein the distribution of parameters for the PK model are associated with an auxiliary variable.

12. The method of claim 11, wherein the auxiliary variable is a biomarker for a patient population.

13. The method of claim 10, wherein simulating the PK model with the distribution of parameters further comprises:
using one or more virtual drug dosages.

14. The method of claim 13, wherein simulating the PK model further comprises:
determining at least a targeted tissue concentration and blood concentrations for each of the one or more virtual drug dosages.

15. The method of claim 14, further comprising:
determining which of the one or more virtual drug dosages is within a tolerance of a patient population.

16. The method of claim 14, further comprising:
providing one or more recommendations based on at least the targeted tissue concentrations and blood concentrations for each of the one or more virtual drug dosages.

17. A computer system for model inversion, comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
training a generator of a generative adversarial network to sample a distribution of input parameters of a mechanistic model, wherein the mechanistic model is a QSP model;
generating a distribution of parameters for the QSP model; and
simulating the QSP model with the distribution of parameters.

18. The computer system of claim 17, wherein the distribution of parameters for the QSP model are associated with an auxiliary variable.

19. The computer system of claim 18, wherein the auxiliary variable is a disease metric for a patient population.

20. The computer system of claim 17, wherein simulating the QSP model with the distribution of parameters further comprises:
using one or more virtual drugs.

21. A computer system for model inversion, comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
training a generator of a generative adversarial network to sample a distribution of input parameters of a mechanistic model, wherein the mechanistic model is a PK model;
generating a distribution of parameters for the PK model; and
simulating the PK model with the distribution of parameters.

22. The computer system of claim 21, wherein the distribution of parameters for the PK model are associated with an auxiliary variable.

23. A method for model inversion, the method comprising:
training a generator of a generative adversarial network to sample a distribution of input parameters of a mechanistic model;
generating a distribution of parameters for the mechanistic model;
minimizing a divergence between the distribution of parameters and a prior distribution of parameters; and
simulating the mechanistic model with the distribution of parameters.

24. The method of claim 23, wherein the mechanistic model is simulated with the distribution of parameters based on an acceptable divergence between the distribution of parameters and the prior distribution of parameters.

25. The method of claim 23, wherein the divergence is minimized within a parametric model of parameter density.
